(19) United States
(12) Patent Application Publication
(10) Pub. No.: US 2011/0293541 A1
(43) Pub. Date: Dec. 1, 2011

(54) Oral Care Compositions And Methods Of Making Oral Care Compositions Comprising Silica From Plant Materials

(76) Inventors: John Christian Haught, West Chester, OH (US); Arif Ali Baig, Mason, OH (US); George Endel Deckner, Cincinnati, OH (US); Ian Allan Hughes, Cincinnati, OH (US)

(21) Appl. No.: 13/115,130

(22) Filed: May 25, 2011

Related U.S. Application Data
(60) Provisional application No. 61/348,073, filed on May 25, 2010.
(60) Provisional application No. 61/375,991, filed on Aug. 23, 2010.

(51) Int. Cl. A61K 8/25; A61Q 11/00

(52) U.S. Cl. 424/52; 424/49

(57) ABSTRACT

Embodiments of a method of making an oral care composition comprise providing plant material comprising silica, burning the plant material to remove organic impurities from the plant source material, removing inorganic impurities from the burnt plant material by hydrolyzing with an aqueous acid solution, separating the silica from the burnt plant material, and incorporating the separated silica into an oral care composition.

### CROSS REFERENCE TO RELATED APPLICATIONS

This application claims the benefit of U.S. Provisional Application 61/348,073 filed on May 25, 2010, and U.S. Provisional Application 61/375,991 filed on Aug. 23, 2010, which are incorporated herein by reference.

### BACKGROUND OF THE INVENTION

The present invention is generally directed to oral care compositions and methods of making oral care compositions, and is specifically directed to oral care compositions comprising silica from plant materials.

### SUMMARY OF THE INVENTION

Embodiments of the present invention relate to personal care products (e.g., oral care compositions such as dentrifices, or skin care products, cosmetics, etc) comprising silica obtained from plant materials. Silica (silicon dioxide) may occur in crystalline and amorphous forms. Silica exhibits excellent abrasive characteristics, either alone, or in combination with other types of abrasives in personal care products. A silica abrasive may be used in conventional oral care compositions, for example, dentifrice compositions, in order to remove various deposits from the surface of teeth. Ideally, an effective abrasive material maximizes cleaning while causing minimal abrasion and damage to the hard tooth tissues. Such components ideally are viable as ingredients within dentifrice compositions in terms of compatibility with active components, ability to exhibit rheological modification in formulations for proper dentifrice form (both functionally and aesthetically by the user), and all while simultaneously present in an amount that is cost-effective and having sufficient abrasive and cleaning performance capabilities. While the description herein is generally directed to oral care compositions, it is contemplated that the silica produced by the present methods may be incorporated in various personal care products.

According to one embodiment, a method of making an oral care composition is provided. The method comprises providing plant material comprising silica, burning the plant material to remove organic impurities from the plant material, removing inorganic impurities from the burnt plant material by hydrolyzing with an aqueous acid solution, separating the silica from the burnt plant material, and incorporating the separated silica into an oral care composition.

According to an additional embodiment, an oral care composition is provided. The oral care composition comprises silica derived from plant material, at least one active ingredient, and at least one additional component selected from the group consisting of additional abrasives, buffering agents, water, surfactants, pigments, colorants, dyes, sweeteners, bleaching agents, flavorants, thickening agents, humectants, and mixtures thereof.

Another embodiment provides an oral care composition comprising silica derived from plant material wherein the silica is surface-modified.

These and additional objects and advantages provided by the embodiments of the present invention will be more fully understood in view of the following detailed description, in conjunction with the drawings.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings in which:

FIG. 1 is a flow chart illustrating one embodiment of a method of making an oral care composition according to one or more embodiments of the present invention; and

FIG. 2 is a flow chart illustrating another embodiment of a method of making an oral care composition according to one or more embodiments of the present invention.

### DETAILED DESCRIPTION OF THE INVENTION

As described herein, the present “oral care composition” means a product which in the ordinary course of usage can be retained in the oral cavity for contacting selected dental surfaces and/or oral tissues for purposes of oral activity (e.g., cleaning, whitening, removing plaque, etc.). In addition to cleaning teeth to remove dental plaque, oral care compositions may be used to prevent formation of dental calculus and disorders such as caries, periodontitis and gingivitis, and also to eliminate and prevent oral malodor or halitosis and staining. Some examples of oral care products are toothpastes (including single or multi phase), dentifrices, tooth gels, subgingival gels, foams, mouthrinses, denture products, mouthsprays, lozenges, chewable tablets or chewing gums and strips or films for direct application or attachment to oral surfaces including any hard or soft oral tissues. The oral care composition may be a single-phase oral care composition or may be a combination of two or more oral compositions delivered in various phases. Typically, the oral composition is a product that is retained in the oral cavity for a time sufficient to contact substantially all of the dental surfaces and/or oral tissues for purposes of oral activity (e.g., cleaning, whitening, removing plaque, etc.).

The term “dentifrice,” as used herein, means paste, gel, powder, or liquid formulations unless otherwise specified, used to treat the surfaces of the oral cavity. The dentifrice composition may be a single phase composition or may be a combination of two or more separate dentifrice compositions. The dentifrice composition may be in any desired form, such as deep striped, surface striped, multilayered, having the gel surrounding the paste, a sheath/core arrangement, a co-extruded sheath/core arrangement, or any combination thereof. Each dentifrice composition in a dentifrice comprising two or more separate dentifrice compositions may be contained in a physically separated compartment of a dispenser and dispensed side-by-side or may be striped without physical separation.

The term “plant material” refers to solid biomass vegetation containing silica levels of greater than 0.02% by weight, for example, non-wood cellulosic, hemi-cellulosic and lignin crops, such as cereal grains; rice husks, rice straw, rye straw, cereal straw, perennial grasses, leafy portion of root crops (beets and turnips), sugarcane, corn stalks, and marine biomass (algae, kelp, and seaweed), and sea buckthorn (Hippophae rhamnoides). Other non-vegetable silica sources include oil palms, two species of the Arecaceae palm family (kernels, fleshy pericarp, or the plant leaves), date palms (Phoenix dactylifera), semi-rigid plant material such as bamboo and sea buckthorn (Hippophae rhamnoides), and other plants from the woody perennial evergreen plants in the true grass family Poaceae. Grasses may include wheat grass, perennial rhizomatous grass (PRG), miscanthus, reed canarygrass, giant reed grass, switchgrass, or combinations thereof. Plant material may also include any cellulosic, hemi-cellulosic and lignin material used in pulp making industry; or any other non-wood biomass material used as a fuel source. Plant material may also include biogenic silica product obtained from diatomite.

The term “substantially non-hydrated” as used herein means that the material has a low number of surface hydroxyl groups or is substantially free of surface hydroxyl groups. It may also mean that the material contains less than about 5% total water (free or/and bound).

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein. All molecular weights as used herein are weight average molecular weights expressed as grams/mole, unless otherwise specified.

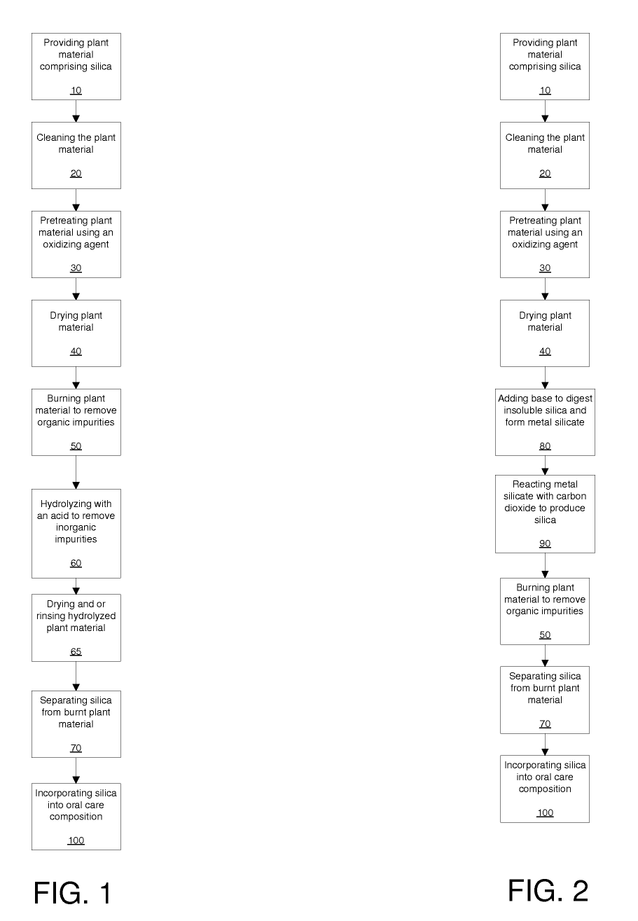

Embodiments of the present invention are generally directed to oral care compositions comprising silica derived from plant material (see step 10 of FIGS. 1 and 2), and methods of removing silica from the plant material for incorporation into the oral care compositions. According to one embodiment, the plant material is burned to remove organic impurities from the plant material as shown in step 50 of FIGS. 1 and 2. As would be familiar to one of ordinary skill in the art, burning may include various suitable processes, for example, pyrolysis or combustion. Pyrolysis, which does not produce reactions with oxygen like combustion, may be conducted at a temperature of from about 300 to about 800° C., or at an elevated temperature above approximately 500° C., but below the melting point of silica.

Additionally, the method may include the optional steps of cleaning 20 or pre-treating 30 the plant material prior to burning. The plant material may be cleaned, more specifically, washed to remove dirt and contamination. Alternatively, using an aqueous cleaning solution (for example, an aqueous solution containing a surfactant) may enhance wettability of the plant material and also help accelerate absorption of an oxidizing solution. Suitable surfactants may include anionic, cationic, nonionic, zwitterionic, amphoteric and betaine surfactants such as, for example, sodium lauryl sulfate, sodium dodecyl benzene sulfonate, alkali metal or ammonium salts of lauroyl sarcosinate, myristoyl sarcosinate, paltnitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Moreover, the plant material may be cleaned by screening using a suitable sieve or perforated screen.

Additionally, the plant material may be pre-treated 30 in a solution containing an oxidizing agent. This may be accomplished with any number of materials, including but not limited to many chlorates, perchlorates, nitrates, permanganates and certain peroxide compounds. In one embodiment, the plant material may be treated with an oxidizing solution comprising hydrogen peroxide or peracetic acid to remove organic hydrocarbons, for example, long hydrocarbon molecules, of the plant material, such as lignin and cellulose. The remaining shorter organic molecules may be removed during the step of burning. After the pretreatment 30, specifically, the above liquid based pre-treatment steps, the plant material may undergo a drying step 40 prior to burning 50.

For many plant materials, the burning step 50 may not be sufficient to remove all impurities, specifically inorganic impurities. Consequently, the inorganic impurities may be removed from the burnt plant material by hydrolyzing 60 with an aqueous acid solution, wherein the acid may include, but not be limited to, hydrochloric, hydrofluoric, and phosphoric acids. Other acids are also contemplated. The inorganic impurities, which typically remain with the silica in the burnt ash, comprise various metal impurities, for example, various calcium, potassium, and magnesium compounds. Specifically, the plant materials may include a large amount of potassium that interacts with the silica at combustion temperatures. Specifically, the inorganic impurities may comprise metal silicates, such as potassium silicate. After hydrolysis 60, the hydrolyzed plant material may be dried or rinsed 65. Rinsing may be performed with as pure water as is practical, such as de-ionized or even distilled water, with very low iron or heavy metal content, to prevent the water itself from contributing undesirable impurities to the silica.

After the impurities (inorganic and organic) have been removed, the silica from the burnt plant may then be separated 70 from the material or ash. The separation 70 may occur via screening, milling, grinding, or combinations thereof. The milling may occur via ball milling, jet milling, or combinations thereof.

Additional process steps may also be used to increase the yield of silica. For example, the solids remaining after hydrolysis may be treated with a metal hydroxide solution, such as about 5% to about 10% sodium hydroxide. The sodium hydroxide extracts silicic acid from the solids by reacting with the silicic acid to produce sodium silicate, which may be converted to silica as described below.

After the silica is extracted from the plant material, the silica may be incorporated into various products, for example, oral care compositions 100 as described below. Alternative processes and or processing steps may be utilized to generate silica, for example, as shown in FIG. 2. In one exemplary embodiment, the method may utilize the step of adding a base to digest insoluble silica from the plant material to form a metal silicate 80, for example sodium silicate. As would be familiar to one of ordinary skill in the art, anaerobic digestion or aerobic digestion may be used to digest the insoluble silica. Many suitable bases are contemplated herein, for example, sodium hydroxide, sodium carbonate, or combinations thereof. Upon production of the metal silicate, the metal silicate may be reacted with carbon dioxide or an acid (e.g., sulfuric acid or hydrochloric acid) to produce silica 90.

Next, the plant material may undergo burning 50 to remove organic impurities as described above, for example, combustion or pyrolysis, to remove organic impurities from the plant material. At which point, the silica may be separated from the burnt plant material via screening, milling, grinding, or combinations thereof. By producing the metal silicate and converting the metal silicate to silica prior to burning, there may be less inorganic impurities present in the burnt plant material. As a result, an additional hydrolysis step as described above may be utilized; however, it may not be necessary in some instances. It is contemplated to conduct separation of the silica from the plant material after the plant material undergoes burning 50, without hydrolysis. After separation, the silica may be incorporated into an oral care composition.

The burning of the plant material, step 50, may also have the result of modifying the surface of the silica. That is, silica, especially silica created through wet processes, may be known to have a relatively high number of surface hydroxyl groups. It is thought that these surface hydroxyl groups can be problematic when formulating certain oral compositions, for example, by causing compatibility issues. In some embodiments of the present invention, the silica may be surface-modified wherein the surface hydroxyl groups to some degree are removed and/or blocked. One process used to remove or block the surface hydroxyl groups or silanol groups involves heating the silica from about 300° C. to about 800° C., in some embodiments from about 600° C. to about 1200° C., in other embodiments from about 950° C. to about 1200° C., in other embodiments from about 600° C. to about 2000° C., and in still other embodiments, from about 1000° C. to about 2000° C.

Alternative processes may be used to remove and/or block the surface hydroxyl groups, including, but not limited to, chemical dehydroxylation such as silanation, coating, for example with chelants, drying, acid-treating, and/or precipitation of the silica. Surface properties of silica can also be modified by adsorption of polymers, surfactants, or surfactant-polymer mixtures.

For example, chemical dehydroxylation, such as silanation, may involve reacting surface hydroxyls with silanes or organosilanes. Other methods involve reacting the surface hydroxyls with other dehydroxylation agents, for example, alcohols, such as methanol, ethanol, propanol, butanol, or glycerol. The silica is combined with an excess amount of the dehydroxylation agent, preferably in the absence of water. The mixture is then reacted to where some or all of the surface hydroxyl groups on the silica are replaced with a radical from the dehydroxylation agent. Methods suitable for dehydroxylation of silica through chemical or thermal means are described in WO 93/23007, U.S. Pat. No. 5,959,005, U.S. Pat. No. 4,954,532, US 2009/0298982, and US 2007/0191537.

Another method for removing surface hydroxyl groups involves acid-washing the silica. For example, U.S. Pat. No. 3,862,307 describes pretreating the silica with hydrofluoric acid at below a pH of 4. U.S. Pat. No. 5,964,937 describes treating the silica with sulfuric acid. U.S. Pat. No. 5,744,114, U.S. Pat. No. 5,968,470, U.S. Pat. No. 5,624,652 disclose similar acid-washing processes to reduce surface hydroxyl groups.

Other methods of surface modification of the silica include those described in U.S. Pat. No. 7,255,852, U.S. Pat. No. 7,438,895, and U.S. Pat. No. 6,946,119, where active silica is precipitated upon the silica substrate particles, reducing the coated silica's surface area.

Other surface-modified silicas and methods to produce them are disclosed in U.S. Pat. No. 6,379,654, U.S. Pat. No. 4,528,181, U.S. Pat. No. 4,575,456, U.S. Pat. Nos. 5,989,524, 5,616,316, and WO 94/06868.

Obviously, combinations of the methods described to remove surface hydroxyls may be used. For example, silica may be acid-washed and then silanated, thereby further reducing the number of surface hydroxyl groups.

In some embodiments, the amount of surface hydroxyl groups may be reduced by about 20%, 30%, 40%, 50%, 60%, 70%, 80%, or 90% from an untreated or unmodified silica. In some embodiments, the surface-modified silica may be considered substantially non-hydrated, meaning that the silica may have a low number of surface hydroxyl groups or is substantially free of surface hydroxyl groups. Surface-modified silicas with less than about 5% bound and free water may be considered substantially non-hydrated.

The amount of surface hydroxyl groups may be measured in any appropriate way, including, but not limited to, calculation of the total bound and free water, measurement of the silanol density, or measurement of a silica's ability to absorb methyl red from a solution.

The total bound and free water can be calculated by totaling two measurements, loss on drying (LOD) and loss on ignition (LOI). For loss on drying, performed first, a sample may be dried at 105° C. for two hours, the weight loss being the free water. For loss on ignition, the dried sample then may be heated for one hour at 1000° C., the weight loss being the bound water. (For another test method, see the United States Pharmacopeia-National Formulary (USP-NF), General Chapter 731, Loss on Drying and USP-NF, General Chapter 733, Loss on Ignition.)

The accounting of surface hydroxyl groups can also be found by using nuclear magnetic resonance spectroscopy (nmr) to measure the silanol density of a particular silica. Silanols are compounds containing silicon atoms to which hydroxy substituents bond directly. When a solids nmr analysis is performed on various silicas, the silicon signal is enhanced by energy transfer from neighboring protons. The amount of signal enhancement depends on the silicon atom's proximity to protons found in the hydroxyl groups located at or near the surface. Therefore, the silanol density, stated as normalized silanol signal intensity (intensity/g), is a measure of surface hydroxyl concentration. Test method for silanol density uses solid state nmr with cross polarization with magic angle spinning (5 kHz) and high power gated proton decoupling and Varian Unity Plus-200 spectrometer with a 7 mm supersonic dual channel probe made by Doty Scientific. The relaxation delay is 4 seconds (s) and the contact time is 3 ms. Number of scans is between 8,000 and 14,000, and the experimental time frame is 10-14 hours per sample. Samples are weighed to 0.1 mg for normalization procedure. Spectra are plotted in absolute intensity mode and integrals are obtained in absolute intensity mode. Silanol density is measured by plotting and integrating spectra in absolute intensity mode.

The surface reactivity of silica, a reflection of the relative number of surface hydroxyls, may additionally be measured by a silica's ability to absorb methyl red from a solution. This measures the relative number of silanols. The test is based on the fact that methyl red will selectively absorb on the reactive silanol sites of a silica surface. The absorbance may be measured at 470 nm. Ten grams of 0.001% methyl red in benzene is added to 0.1 gram each of two silica samples and mixed for five minutes on a magnetic stirrer. The resulting slurries are centrifuged for five minutes at 12,000 rpms, and then the percent transmission at 470 nm is determined for each sample and averaged. See “Improving the Cationic Compatibility of Silica Abrasives Through the Use of Topochemical Reactions” by Gary Kelm, Nov. 1, 1974, in Iler, Ralph K., The Colloid Chemistry of Silica and Silicates, Cornell University Press, Ithaca, N.Y., 1955.

The amount of surface-modified silica from plant material used in the present invention may be from about 1%, 2%, 5%, 7%, 10%, 12%, 15%, 20%, 25%, 30%, 35%, 40%, 45%, 50% to about 5%, 7%, 10%, 12%, 15%, 20%, 25%, 30%, 35%, 40%, 45%, 50%, 55%, 60%, 65%, or 70%, or any combination thereof. The surface-modified silicas of the present invention may be used alone or with other abrasives. A composition may comprise more than one type of surface-modified silica. Another embodiment of the composition may comprise pre-treatment of silica with adsorbates including orally acceptable surfactants, polymers and mixtures thereof followed by mixing with cationic ingredients in the formula. Another embodiment of the composition is in-situ surface modification by adding the surfactants, polymers and mixture in the same mix as the cationic ingredient. The adsorbates coat the surface silanol groups and prevent interaction with cationic ingredients.

Without being bound by theory, it is believed that the surface-modified silica, due to its low number of surface hydroxyl groups, will be less reactive than unmodified silica. Consequently, the surface-modified silica may adsorb less of other components, such as flavors, actives, or cations, leading to better availability for these other components. For example, dentifrices incorporating surface-modified silica may have superior stability and bioavailability for stannous, fluoride, zinc, other cationic antibacterials, and hydrogen peroxide. Surface-modified silica formulated in a dentifrice composition may result in at least about 50%, 60%, 70%, 80%, or 90% compatibility with cations or other components. In some embodiments, the cation may be stannous. In general, cation compatibility may be determined by the “% CPC compatibility test” disclosed in U.S. Pat. No. 7,255,852.

The step of surface-modifying the silica may be done during the burning step 50 or immediately after the burning step 50 if done by processes other than heating. Alternatively, the surface-modification, by heating or by any of the processes described above, may be done after step 70, after the silica has been separated from the burnt plant material and before incorporating the silica into an oral care composition.

In addition to the abrasive component, the oral care composition (e.g., dentifrice) may also include active components (such as fluoride), humectants to provide proper suspension and delivery of the oral care product, pH buffering agents, bleaching agents, sweeteners, surfactants, flavorants, pigments, colorants, dyes, thickening agents, and mixtures thereof.

In addition to the silica extracted from the plant material, as noted above, the oral composition may include the present silica along with other abrasive materials. Combining the plant derived silica with other dental abrasives will provide potential advantages by furthering the Pellicle Cleaning Ratio (PCR) to at least 80 and greater than 100. As would be familiar to one or ordinary skill in the art, the PCR test measures the ability of a dentifrice composition to remove pellicle film from a tooth under fixed brushing conditions.

The additional abrasive materials may include but not limited to precipitated silica, precipitated and ground calcium carbonate, sodium bicarbonate, aluminum oxides, zeolites, dicalcium phosphate, dicalcium phosphate dihydrate, calcium metasilicate, calcium pyrophosphate, alumina, calcined alumina, aluminum silicate, chalk, bentonite, particulate thermosetting resins and other suitable abrasive materials known to a person of ordinary skill in the art, may be introduced within the desired abrasive compositions to tailor the polishing characteristics of the target formulation. The abrasives, when incorporated into dentifrice compositions, are present at ranges from about 0.1 to about 25%, depending upon the application. In exemplary embodiments, the oral care composition may comprise about 1 to about 10% silica derived from plant material and from about 0.1 to about 10% of an additional abrasive. In further exemplary embodiments, the ratio of additional abrasive by weight to the present silica by weight is from about 1:20 to about 20:1, or from about 1:5 to about 5:1. The following table provides further compositional examples and ratios demonstrating the amounts of plant derived silica and additional abrasives added to the oral care composition.

| | Range of Additional | | | | | | |
|---|---|---|---|---|---|---|---|
| Additional | Abrasive to Present | | | | | | |
| Abrasive | Silica | Example 1 | Example 2 | Example 3 | Example 4 | Example 5 | Example 6 |
| Abrasive silica | 1:1 to 0.2:1 | 1:1 | 0.75:1 | 0.5:1 | 0.4:1 | 0.3:1 | 0.2:1 |
| AlO3 | 0.5:1 to 0.05:1 | 0.5:1 | 0.4:1 | 0.3:1 | 0.2:1 | 01:1 | 0.05:1 |
| ZnO | 0.5:1 to 0.05:1 | 0.5:1 | 0.4:1 | 0.3:1 | 0.2:1 | 01:1 | 0.05:1 |
| CaCO2 | 0.25:1 to 10:0.5 | 0.25:1 | 0.5:1 | 1:1 | 2:1 | 5:1 | 10:0.5 |
| NaCO2 | 0.25:1 to 10:0.5 | 0.25:1 | 0.5:1 | 1:1 | 2:1 | 5:1 | 10:0.5 |
| Zeolite | 0.5:1 to 0.05:1 | 0.5:1 | 0.4:1 | 0.3:1 | 0.2:1 | 01:1 | 0.05:1 |
| Thickening | 0.25:1 to 10:0.5 | 0.25:1 | 0.5:1 | 1:1 | 2:1 | 5:1 | 10:0.5 |
| Silica | | | | | | | |

Depending on the application, it may be desirable to modify the hardness and the particle size for the silica particles. In one or more exemplary embodiments, the median particle size of the plant derived silica may be in the range of about 2 to about 50 microns or from about 4 to about 15 microns.

The compositions of the present invention may also comprise one or more oral care active ingredients. While not being limited, the active ingredients are directed to treating or preventing structural problems for teeth, plaque, calculus, cavities, inflamed and/or bleeding gums, gingivitis, fungal infections such as Candida, mucosal wounds, lesions, ulcers, aphthous ulcers, cold sores, tooth abscesses, and the elimination of mouth malodor resulting from the conditions above and other causes such as microbial proliferation. As would be familiar to one of ordinary skill in the art, the active ingredients of the present invention may be cationic agents and antibacterials, essential oils, stain-control agents, chelants, high-molecular weight polyethylene oxide and poloxamers, and/or desensitizing agents, as described in US publication 2010/0135924.

Actives may also be anti-plaque agents, anti-inflammatory agents, nutrients, whitening or bleaching agents, antioxidants, antiviral actives, antimicrobial agents, H-2 antagonists, desensitizing agents, and combinations thereof, as described in US publication 2010/0135924. Suitable oral care actives include any material that is generally considered safe for use in the oral cavity and that provides changes to the overall appearance and/or health of the oral cavity, including oral cavity surfaces (e.g., teeth) and tissues (e.g., gums). When present, the level of oral care active is present in the amount of from about 0.001% to about 90%, by weight of the composition, in one embodiment from about 0.01% to about 50%, by weight of the composition, in another embodiment from about 0.1% to about 30%, by weight of the composition.

Suitable active ingredients may include those components shown and described in U.S. Pat. No. 6,509,007, which are incorporated by reference herein in their entirety. The active ingredients may also comprise fluoride ion sources or fluoride ion-yielding materials shown and described in U.S. Pat. Nos. 3,535,421, 3,678,154, 4,994,262, and 6,509,007, as well as U.S. Publication U.S. 20050143274, which are all incorporated by reference herein in their entirety. The active ingredients may also include a stannous ion source, wherein the stannous ions may include stannous fluoride and/or other stannous salts, such organic stannous carboxylates, such as stannous acetate, stannous gluconate, stannous oxalate, stannous malonate, stannous citrate, stannous ethylene glycoxide, stannous formate, stannous sulfate, stannous lactate, stannous tartrate, stannous halides such as stannous chlorides, stannous bromide, stannous iodide and stannous chloride dihydrate. Other actives include a copper ion source, a strontium ion source, cetylpyrimidium chloride, and chlorhexidine.

The compositions of the present invention may also comprise an orally-acceptable carrier, as described in US publication 2010/0135924.

Humectants may also be included in the oral composition to add body or “mouth texture” as well as preventing the dentifrice from drying out. Suitable humectants include, but are not limited to, polyethylene glycol (at a variety of different molecular weights), propylene glycol, glycerin (glycerol), erythritol, xylitol, sorbitol, mannitol, lactitol, diethylene glycol monoethyl ether, polyethylene sorbitan monolaurate, polysorbate, and hydrogenated starch hydrolyzates, as well as mixtures, of these compounds. Humectants may be present in an amount from about 0.50% to about 45% by weight of the composition.

Sweeteners may also be added to impart a pleasing taste to the oral care composition and/or product. Suitable sweeteners include, but are not limited to, saccharin (as sodium, potassium or calcium saccharin), cyclamate (as a sodium, potassium or calcium salt), xylitol, sorbitol, acesulfane-K, thaumatin, neohisperidin dihydrochalcone, ammoniated glycyrrhizin, dextrose, levulose, sucrose, mannose, and glucose. The sweetening agent may be present in an effective amount from about 0.05% to about 2.5% by weight of the composition.

Sensates may be added to the present compositions. The term “sensate” as used herein refers to a material in which its predominant effect in the oral cavity is to impart a sensation, for example, a taste, moisturization, warming, cooling, and/or tingling sensation. A sensate may be, but is not limited to, a flavor, a sweetener, a coolant, a saliva stimulant, or a TRPV1 activator. Coolants suitable for the present compositions include the paramenthan carboxyamide agents such as N-ethyl-p-menthan-3-carboxamide (known commercially as WS-3, WS-23, WS-5), MGA, TK-10, Physcool, and mixtures thereof. Other coolants may include those listed in US 2008/0008660. Saliva stimulants, or sialagogues, such as pellitorin, may be used. Saliva stimulating agents are further disclosed in U.S. Pat. No. 4,820,506. Sensates are generally used in the oral care compositions at levels of from about 0.001% to about 5%, by weight of the oral care composition.

Surfactants may optionally be used in the compositions of the present invention. The surfactant may be a detersive material which imparts to the composition detersive and foaming properties. Suitable surfactants may include, but are not limited to, anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, paltnitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. The surfactant may be present in the exemplary oral care compositions of the present invention in an amount from about 0.1 to about 15% by weight, preferably from about 0.3% to about 5% by weight, such as from about 0.3% to about 2%, by weight.

Flavoring agents optionally can be added to the oral care compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds. Flavoring agents may be present in an effective amount from about 0.5% to about 20% by weight of the composition.

Colorants may be added to improve the aesthetic appearance of the oral care composition and/or product. Suitable colorants may be selected from colorants approved by appropriate regulatory bodies such as the FDA and those listed in the European Food and Pharmaceutical Directives and include, but are not limited to, pigments, such as TiO₂, and colors, such as FD&C and D&C dyes. Colorants may be present in an effective amount from about 0.1% to 20% with respect to the by weight of the composition.

Thickening agents may also be useful to increase retention of the composition on the teeth. Suitable thickening agents include, but are not limited to, starch, glycerite of starch, gums such as gum karaya (sterculia gum), gum tragacanth, gum arabic, gum ghatti, gum acacia, xanthan gum, guar gum and cellulose gum, magnesium aluminum silicate (Veegum), carrageenan, sodium alginate, agar-agar, pectin, gelatin, cellulose compounds such as cellulose, carboxymethyl cellulose, hydroxyethyl cellulose, hydroxypropyl cellulose, hydroxymethylcellulose, hydroxymethyl carboxypropyl cellulose, methyl cellulose, ethyl cellulose, and sulfated cellulose, natural and synthetic clays such as hectorite clays, carbomers, as well as mixtures of these compounds. Thickening agents or binders may be present in an amount from about 0 wt % to about by 15% weight of an oral care composition.

In another embodiment, the thickening agent can be an associative thickener or stabilizer, such as a hydrophobically modified alkali soluble acrylic emulsion or a hydrophobically modified nonionic polyol polymer, i.e., a hydrophobically modified urethane polymer, hydrophobically modified ethoxylated urethane polymer or combinations thereof. Associative thickeners may increase the retention or adhesion of compositions herein on the tooth surfaces, may slow the erosion of the compositions once applied on the tooth surfaces, and may improve the release of the compositions from the optional release liner disclosed herein.

Preservatives may also be optionally added to the exemplary oral care compositions of the present invention to prevent bacterial growth. Suitable preservatives may include, but not be limited to, preservatives approved for use in oral compositions such as methylparaben, propylparaben and sodium benzoate, phenyl mercuric nitrate, sodium bisulfate, disodium calcium EDTA, chlorobutanol, etc and mixtures thereof. The preservatives may be present in an amount from about 0.5% to about 5.0% by weight of the composition.

PH adjusting or buffering agents may also be utilized in the present composition. Suitable pH buffering agents may include, but not be limited to, alkalis such as sodium hydroxide, ammonium hydroxide, monosodium phosphate, dibasic sodium phosphate, trisodium phosphate, sodium bicarbonate and similar compounds that are capable of raising the pH of the composition between about 5.5 and about 14.

Water may provide the balance of the oral care composition in addition to the additives mentioned. In some embodiments, the water may be deionized and free of impurities. The oral care composition will usually comprise from about 0 to about 60 wt % of water, or from about 5 to about 35%, or from between about 20 wt % to about 35% by weight of the composition.

### Non-Limiting Examples

The dentifrice compositions illustrated in the following examples illustrate specific embodiments of the dentifrice compositions of the present invention, but are not intended to be limiting thereof. Other modifications can be undertaken by the skilled artisan without departing from the spirit and scope of this invention.

| | Formula A | Formula B | Formula C |
|---|---|---|---|
| Ingredient | Percent | Percent | Percent |
| Water, USP | 30.000 | 30.000 | 30.000 |
| Sodium Gluconate | 1.064 | 1.064 | 1.064 |
| SnF2, USP | 0.454 | 0.454 | 0.454 |
| Zinc Lactate | 0.670 | 0.670 | 0.670 |
| Sorbitol(LRS) USP | 40.162 | 41.862 | 39.932 |
| Gantrez S 95 | 2.000 | — | — |
| Gantrez S-97 | — | — | 1.000 |
| Sodium Tripolyphosphate | — | — | 0.930 |
| Phytic acid | — | 0.800 | 0.800 |
| Sodium lauryl sulfate solution (28%) | 5.000 | 5.000 | 5.000 |
| Essential oil blend | 1.000 | — | — |
| Plant-derived silica, (surface- | 15.000 | 15.000 | 15.000 |
| modified) | | | |
| Hydroxyethyl cellulose (HEC | 0.500 | 0.500 | 0.500 |
| Natrasol 250M) | | | |
| Polyethylene Oxide (MW 100,000- | 0.500 | — | — |
| 10,000000) | | | |
| NaOH (50% soln) | — | 1.000 | 1.000 |
| CMC 7M8SF | 1.300 | 1.300 | 1.300 |
| Carrageenan mixture | 0.700 | 0.700 | 0.700 |
| Saccharin Sodium | 0.800 | 0.800 | 0.800 |
| Flavor - Tea Berry | 0.650 | 0.650 | 0.650 |
| FDC Blue | 0.200 | 0.200 | 0.200 |

It is further noted that terms like “preferably,” “generally,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is additionally noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it will be obvious to those skilled in the art that various changes and modifications may be made without departing from the spirit and scope of the invention. It is therefore intended to cover in the appended claims all such changes and modifications that are within the scope of the invention.

What is claimed is:

1. A method of making an oral care composition, comprising:
providing plant material comprising silica;
burning the plant material to remove organic impurities from the plant material;
removing inorganic impurities from the burnt plant material by hydrolyzing with an aqueous acid solution;
separating the silica from the burnt plant material; and
incorporating the separated silica into an oral care composition.

2. The method of claim 1, wherein the plant material comprises straw of cereal grains.

3. The method of claim 1, wherein the plant material comprises rice straw, wheat straw, or combinations thereof.

4. The method of claim 1, further comprising cleaning the plant material prior to burning.

5. The method of claim 1, further comprising pre-treating the plant source material with an oxidizing solution prior to the burning step.

6. The method of claim 5, further comprising drying the pre-treated plant material prior to the burning step.

7. The method of claim 1, wherein the burning step occurs via pyrolysis or combustion.

8. The method of claim 1, wherein the burning step results in surface modification of the silica.

9. The method of claim 1, further comprising the step of surface-modifying the silica after separating the silica from the burnt plant material and before incorporating the silica into an oral care composition.

10. The method of claim 9, wherein the surface-modifying of the silica is done by a process selected from the group consisting of heating, chemical dehydroxylation, coating, drying, acid-treating, precipitation of the silica, and adsorption of polymers, surfactants, or surfactant-polymer mixtures.

11. The method of claim 1, wherein burning the plant material is done from about 600° C. to about 2000° C.

12. An oral care composition, comprising:
silica derived from plant material;
at least one active ingredient; and
at least one additional component selected from the group consisting of additional abrasives, buffering agents, water, surfactants, pigments, colorants, dyes, sweeteners, bleaching agents, flavorants, thickening agents, humectants, sensates, and mixtures thereof.

13. The oral composition of claim 12, wherein the silica derived from plant material is surface-modified.

14. The oral composition of claim 13, wherein the surface-modification of the silica is done by a process selected from the group consisting of heating, chemical dehydroxylation, coating, drying, acid-treating, precipitation of the silica, and adsorption of polymers, surfactants, or surfactant-polymer mixtures.

15. The oral composition of claim 13, wherein the silica is surface-modified by heating from about 600° C. to about 2000° C.

16. The oral composition of claim 13, wherein the silica is surface-modified by heating from about 950° C. to about 1200° C.

17. The oral care composition of claim 13, wherein the active ingredient is selected from the group consisting of antibacterials, stain-control agents, desensitizing agents, anti-inflammatory agents, and mixtures thereof.

18. The oral care composition of claim 13, wherein the active ingredient is selected from the group consisting of a cationic agent, a blend of at least two essential oils, a stain-control agent, a desensitizing agent, a polyethylene oxide with a molecular weight from about 200,000 to about 7,000,000, and mixtures thereof.

19. The oral care composition of claim 18, wherein the cationic agent is selected from the group consisting of a stannous ion source, copper ion source, strontium ion source, cetylpyrimidium chloride, chlorhexidine, and mixtures thereof.

20. The oral care composition of claim 13, wherein the plant material comprises straw of rice cereal.
